(19) United States
(12) Patent Application Publication
(10) Pub. No.: US 2025/0018216 A1
(43) Pub. Date: Jan. 16, 2025

(54) BODY IRRADIATION DEVICE FOR USE OF DIRECTIONAL ACTINIC RADIATION ON A LIVING ORGANISM

(71) Applicant: KBL GmbH, Dernback (DE)

(72) Inventor: Ulrich Obel, Dachsenhausen (DE)

(21) Appl. No.: 18/719,248

(22) PCT Filed: Aug. 2, 2022
(86) PCT No.: PCT/EP2022/071719

§ 371 Date: Jun. 12, 2024

(30) Foreign Application Priority Data
Aug. 16, 2021 (DE) 202021104364.4
Sep. 7, 2021 (DE) 202021104807.7

(51) Int. Cl. A61N 5/06

(52) U.S. Cl. CPC A61N 5/0614; A61N 2005/0615; A61N 2005/0651; A61N 2005/0661

(57) ABSTRACT

The invention relates to a body irradiation device for use of directional actinic radiation on a living organism, in particular a human being, which comprises at least one irradiation module, and wherein the at least one irradiation module comprises: at least two LED radiation sources which generate the actinic radiation and are arranged on a common carrier; a plate which spans the at least two LED radiation sources, wherein the plate is spaced apart from the carrier; at least two planoconvex optical lenses which are integrally bonded to the plate such that the flat surfaces of the lenses face the carrier, and wherein each lens is configured and arranged in such a way as to at least substantially collimate or direct radiation emitted by one of the LED radiation sources.

### OBJECT OF THE INVENTION

The invention relates to a body irradiation device for use of directional actinic radiation on a living organism, in particular a human being, which comprises at least one irradiation module and wherein the at least one irradiation module comprises at least two LED radiation sources.

### PRIOR ART

There are known body irradiation devices for the body of a living organism, in particular human beings, which are in particular designed in the form of a tanning booth having a bed surface or a stand-up tanner or a red light treatment table in which the body or parts of the body are exposed to a spectrum of radiation at specific wavelength ranges in order to influence cosmetic aspects of the body, the well-being, health or regeneration of the body or being.

In general, such body irradiation devices thereby use low-pressure radiant tubes, high-pressure radiant tubes or even high-pressure radiation lamps. In recent years, LED light sources have also been increasingly used in body irradiation devices.

Purely by way of example, reference is made in this regard to the DE 20 2021 100 716 U1 document which relates to a body irradiation device for irradiating a body of a person or a part of a person's body, in particular with cosmetically/hygienically beneficial radiation. The body irradiation device comprises a radiation source with a base and at least one first LED chip able to emit a first radiation spectrum having a first radiation peak and at least one second LED chip able to emit a second radiation spectrum having a different radiation peak from the first radiation peak, wherein the first LED chip and the second LED chip are arranged under a common lens in an LED housing and separately controllable.

### Task

The task of the invention is that of providing an improved body irradiation device. A particular task of the invention is providing large-area irradiation from LED radiation sources in the improved body irradiation device.

This task is solved by the teaching of claim 1. Advantageous embodiments are claimed in subclaims.

### DESCRIPTION

One aspect of the invention relates to a body irradiation device for use of directional actinic radiation on a living organism, in particular a human being, which comprises at least one irradiation module and wherein the at least one irradiation module comprises:
at least two LED radiation sources which generate the actinic radiation and are arranged on a common carrier;
a plate which spans the at least two first LED radiation sources;
spacers between the plate and the carrier which keep the plate and the carrier at a defined distance;
at least two planoconvex optical lenses which are integrally bonded to the plate such that the flat surfaces of the lenses face the carrier, and wherein each lens is configured and arranged in such a way as to at least substantially collimate or direct radiation emitted by one of the LED radiation sources.

A body irradiation device in the sense of the invention is preferably configured to irradiate at least part of the surface of the living organism's body.

The term “actinic radiation” within the meaning of the invention denotes light or (broader) radiation of the overall electromagnetic spectrum which has a photochemical (including photobiochemical) effect and can potentially include light/radiation of natural or artificial origin (see the definition in “Römpp Chemie-Lexikon” {Römpp's Chemistry Lexicon}, Thieme Verlag publishers, Stuttgart, Germany). In the claims and the description, “actinic light” or “actinic radiation” is used for light or radiation of artificial origin, preferably light/radiation emitted by radiation sources in a body irradiation device. The term “directional actinic radiation” as used within the present description and claims denotes actinic radiation which is radiated with a preferential, if not more or less exclusive, focus at a target which, as per the present invention, is a living organism, preferably a human being.

A lens in the sense of the invention is an optical element. Preferably, at least one of the two surfaces is—spheroidal or spherical—curved. Further preferably, the lens can be designed as a prisma.

In one preferential embodiment of the body irradiation device, which can be implemented separately or together with one or two or more or all of the other features of the invention, the actinic radiation can be actinic radiation in a broad wavelength range. Alternatively, albeit also preferentially, the actinic radiation can be actinic radiation within a narrow wavelength range or even actinic radiation of a very specific wavelength or multiple specific wavelengths. This is known to the person skilled in the art who is able to select the wavelength(s) or wavelength ranges or bands to use pursuant to the requirements of the individual case.

Preferable examples of actinic radiation with applicable wavelengths within the scope of the present invention are as follows:
actinic radiation with a wavelength λ in the range of 280 to 315 nm (UV-B radiation) and/or actinic radiation with a wavelength λ in the range of 315 to 400 nm (UV-A radiation) for tanning the human body; i.e. for activating the formation and conversion of melanin into the dark (brown) form of the pigment naturally occurring in human skin, the tanning of which can serve purposes in the wellness and/or cosmetic and/or medical fields; actinic radiation in the form of short-wave UV-B radiation, in particular in the wavelength range λ of approximately 285 nm to 305 nm for promoting vitamin D biosynthesis from vitamin D precursors in the human skin; or actinic radiation with wavelengths λ in the visible and near-IR (infrared) range (400 or better>550 nm to 850 nm), which actinic radiation, when applied to the human skin, stimulates biosynthesis of useful compounds for the nourishment, rejuvenation and regeneration of the skin such as, for example, collagen, elastin, keratin and hyaluronic acid.

A carrier in the sense of the invention is preferably a substrate and/or a printed circuit board.

A red spectrum in the sense of the invention preferably denotes visible red radiation and/or infrared radiation.

The invention is based on the awareness that when using LED radiation sources in body irradiation devices, a large number of LED light sources are necessary to ensure uniform irradiation of the organism over larger areas. This follows from, on the one hand, the radiation angle of the LED radiation waves needing to be limited by a collimator in order to achieve uniform radiation intensity on the body of the organism but yet, on the other hand, collimated or respectively directed radiation only being able to irradiate a smaller area due to the reduced radiation angle.

LED sources are thereby intended to replace the low-pressure tubes or high-pressure lamps generally used in body irradiation devices for irradiating large areas. One advantage of LED radiation sources is that they operate more energy efficiently than tubes and in particular generate less waste heat that needs to be dissipated. Moreover, LED radiation sources are more durable than tubes such that the body irradiation devices require less maintenance, which leads to substantial cost savings, particularly in the commercial sector.

Lastly, using LED radiation sources enables a defined radiation spectrum and a defined radiation intensity to be generated anywhere such an LED radiation source is located. This is of great advantage compared to tubes which have different radiation intensities over their length. Particularly their ends experience a decreasing intensity of radiation. This is disadvantageous since it is precisely these regions of the tubes which irradiate the foot and head area of a human being, areas in which users of body irradiation devices generally desire particularly strong tanning. Using LED radiation sources, particularly in these areas, can remedy this.

By using a plate to which several lenses are affixed, the individual lenses and thus also the different LED radiation sources can be arranged relatively close together such that uniform radiation intensity can be achieved on the area irradiated. Particularly compared to irradiation modules which use reflector collimators, this thereby enables significantly increasing the density of LED radiation sources.

At the same time, however, the irradiation modules can be made comparatively large since the plate supported on the carrier via the spacers can span a large area of the carrier on which the LED radiation sources are located.

The invention thus succeeds in combining two optimization goals previously considered incompatible when using LED radiation sources in body irradiation devices, namely, on the one hand, ensuring uniform irradiation intensity on the irradiated surface and, on the other hand, being able to irradiate a comparatively large area with one irradiation module.

A further advantage of the invention lies in the fact of the LED radiation sources being able to be arranged on a common carrier. This is particularly efficient and economical and also increases the overall stability of the irradiation module. This also enables it to be made comparatively large.

Preferably, the carrier is a thereby a printed circuit board or even the substrate for the LED radiation sources. In both cases, the carrier with the LED radiation sources can be manufactured particularly cost-effectively compared to LED radiation sources arranged on separate circuit boards and/or substrates.

By using the plate arranged at a defined distance from the carrier in conjunction with the carrier, a particularly simple structure without reflector collimators can furthermore be realized. This in particular leads to savings in materials and in the manufacturing process. In addition, contaminants such as dust particles can accumulate on reflectors, which can affect radiation efficiency.

In one advantageous embodiment of the body irradiation device, the opposite side of the plate from the carrier is satinized. The satinizing scatters radiation that passes through the plate outside the area of the lenses. This enables reducing or preferably preventing irregularities in the irradiation intensity of the irradiated area due to directed radiation emission outside of the lenses.

Due to the integral bonding, satinizing can be eliminated in the area of the lenses if the flat surfaces of the lenses facing the carrier rest on the plate. In particular, the use of an adhesive ensures a filling of the unevenness on the plate created by the satinizing and the plate thus being transparent again at this point. The radiation can therefore pass through the plate unhindered in the area of the lenses.

In a further advantageous embodiment of the body irradiation device, one side of the plate, in particular the side of the plate facing the carrier, is coated, particularly with a mirroring, in particular one reflective of radiation in the visible spectrum. The mirroring can even further reduce the emission of directed radiation outside the areas of the lenses.

In a further advantageous embodiment of the body irradiation device, the at least two planoconvex lenses are glass-molded lenses. Glass-molded lenses are particularly economical to produce.

In a further advantageous embodiment of the body irradiation device, the plate is a glass plate. A glass plate exhibits high strength coupled with high radiation transmissivity. Furthermore, glass is particularly resistant to radiation, particularly UV radiation.

In a further advantageous embodiment of the body irradiation device, the at least one irradiation module further comprises:
an at least partially transparent plastic plate which covers the plate, in particular on the side facing the carrier, and has recesses in the region of the at least two lenses. By providing a plastic plate, the actual plate can be further reinforced so that it can cover large areas of an irradiation module without needing to be supported.

In a further advantageous embodiment of the body irradiation device, the plate itself is a partially transparent plastic plate. Using a plastic plate is particularly economical. At the same time, the plastic plate can be machined particularly easily, e.g. in order to cut recesses through same.

In a further advantageous embodiment of the body irradiation device, the plastic plate comprises recesses, in particular round recesses, in the region of the at least two lenses which are designed such that the at least two lenses are able to be integrally bonded to the plastic plate. This offers the advantage of, on the one hand, being able to use only one plastic plate and, on the other hand, being able to ensure the passage of high radiation intensity through the lenses via the recesses.

In a further advantageous embodiment of the body irradiation device, the recesses form a seat for the flat side of the at least two lenses. Such a seat can in particular be designed as a retainer or bearing for the flat side of the at least two lenses. Preferably, the seat is thereby formed as a protrusion or a shoulder of or respectively in the recesses. This enables the lenses to be particularly well secured in the recesses and centered/aligned.

In a further advantageous embodiment of the body irradiation device, the plate and/or the plastic plate comprises a fluorescent material and is in particular coated with the fluorescent material. Using fluorescent material can alert a user of the body irradiation device that the respective LED radiation source is emitting radiation in the area of the fluorescent material. This is relevant from a safety standpoint, particularly in the case of UV radiation which can for example damage the human eye

In a further advantageous embodiment of the body irradiation device, a first region of the plate and/or the plastic plate contains violet fluorescent material opposite from a UV-A LED radiation source, wherein a second region of the plate and/or the plastic plate contains red fluorescent material opposite from a red LED radiation source and/or wherein a third region of the plate and/or the plastic plate contains yellow fluorescent material opposite from a UV-B LED radiation source. Providing different types of fluorescent material matched to the respective LED radiation source enables increasing the intensity of the fluorescence. At the same time, the user can be visually informed about the respective type of radiation emitted.

In a further advantageous embodiment of the body irradiation device, the at least two planoconvex lenses are glass lenses. Glass is particularly resistant to radiation, in particular UV radiation. Furthermore, due to its refractive index, glass proves particularly suitable for optical elements.

In a further advantageous embodiment of the body irradiation device, the at least two planoconvex lenses are silicone lenses. This can simplify the manufacturing process, in particular the plate and/or the plastic plate and the lenses can be integrally formed in a single primary forming process. Preferably, the plate in this case is made of silicone and is further preferably supported by the plastic plate.

In a further advantageous embodiment of the body irradiation device, at least one of the LED radiation sources emits UV-A radiation and the lens associated with this LED radiation source for collimating the radiation consists of borosilicate glass. Borosilicate glass is particularly transparent to UV-A radiation, enabling very good energy efficiency with low irradiation module loss to be achieved.

In a further advantageous embodiment of the body irradiation device, at least one of the LED radiation sources emits UV-B radiation and the lens associated with this LED radiation source for collimating the radiation consists of quartz glass. Quartz glass exhibits very good UV-B radiation transmissivity. Using quartz glass in the lens enables minimizing radiation losses at the lens, thereby enabling high energy efficiency of the irradiation module to be achieved.

In a further advantageous embodiment of the body irradiation device, the integral bond is a UV-curing adhesive. The UV-curing adhesive has the advantage of being UV-resistant. As a result, the integral bond is not rendered weaker by exposure to UV radiation but rather even stronger.

In a further advantageous embodiment of the body irradiation device, at least one of the LED radiation sources comprises a first LED chip having a first radiation spectrum and a second LED chip having a second radiation spectrum differing from the first radiation spectrum. As a result, a comparatively broad radiation spectrum band can be realized.

In a further advantageous embodiment of the body irradiation device, at least one first LED radiation source exhibits a first radiation spectrum and at least one second LED radiation source exhibits a second radiation spectrum. This enables different areas with differing radiation spectra to be realized in a single radiation module.

In a further advantageous embodiment of the body irradiation device, the first region of the plate is arranged around a lens which collimates light of the first LED radiation source, or the first LED chip and the second region of the plate is arranged around a lens which collimates light of the second LED radiation source or the second LED chip. This enables the fluorescent material being able to be arranged in different regions of the plate around the respectively corresponding LED radiation source.

In a further advantageous embodiment of the body irradiation device, the first LED chip and/or the first LED radiation source emits a UV-A or UV-B spectrum and the second LED chip and/or the second LED radiation source emits in the red spectrum. In this embodiment, both tanning of the user as well as a deep heating of the user's tissue can occur.

In a further advantageous embodiment of the body irradiation device, the irradiation module further comprises a cooling device configured to cool the carrier at least on the opposite side from the at least two LED radiation sources.

In a further advantageous embodiment of the body irradiation device, same further comprises:
an exposure tunnel capable of surrounding the living organism, wherein the exposure tunnel is formed from at least one lower part of the body irradiation device having a surface made from a material which is substantially transparent to the actinic radiation and at least one upper part of the body irradiation device, wherein the surface separates an inner space, in which the living organism can be exposed to the actinic radiation, from an outer space in which at least one LED irradiation module is mounted in such a way as to emit actinic radiation through the surface, and wherein the upper part also has at least one LED irradiation module mounted in such a way as to emit actinic radiation into the inner space.

Further advantages and features will become apparent from the following description of embodiments in conjunction with the figures. Thereby shown, at least in part schematically:

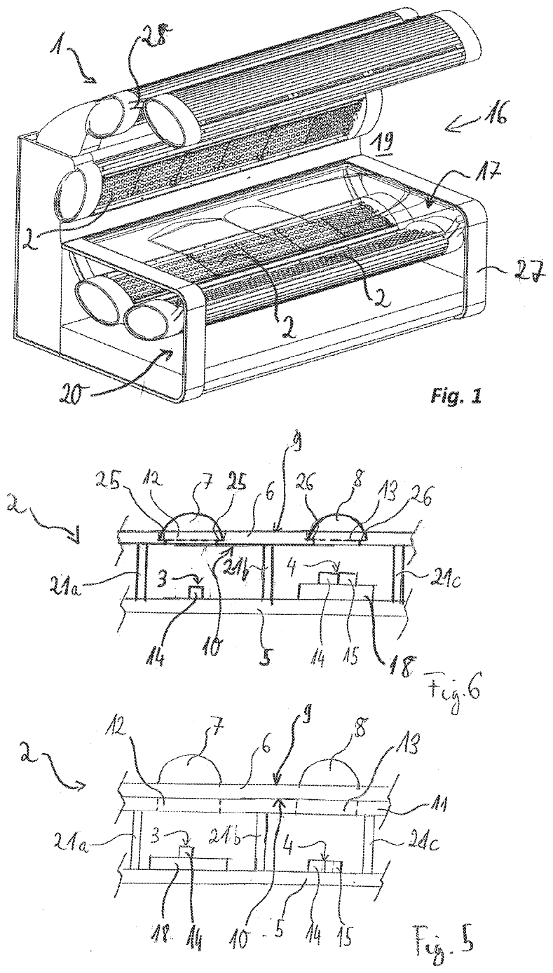

FIG. 1 one embodiment of a body irradiation device;

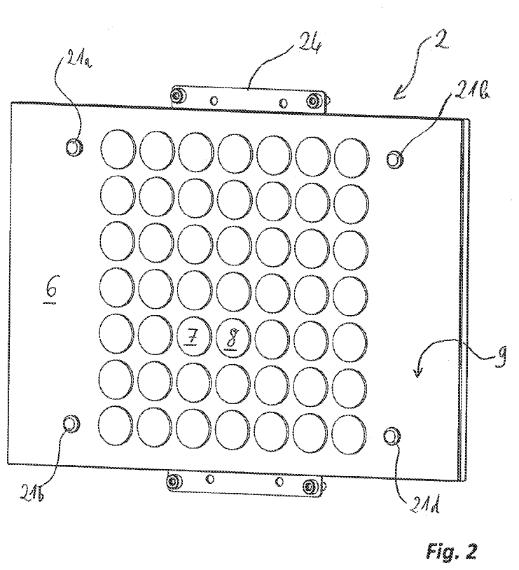

FIG. 2 a top plan view of one embodiment of an irradiation module;

FIG. 3 an exploded view of the irradiation module from FIG. 2;

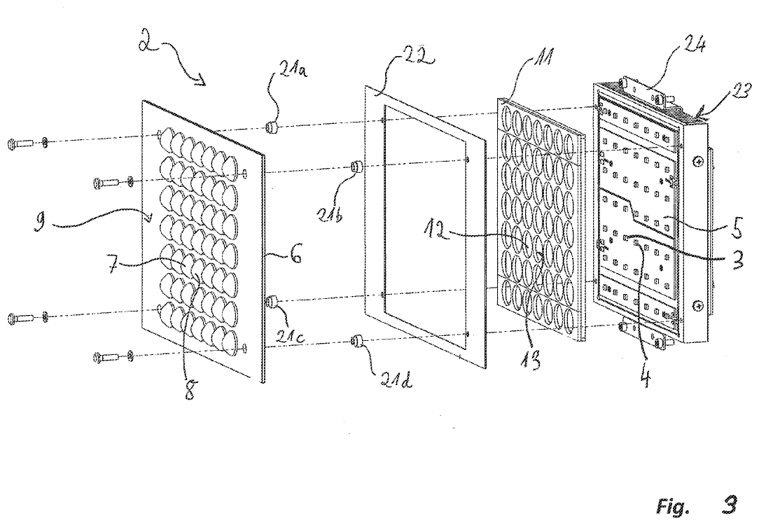

FIG. 4 a further exploded view of components of the carrier of an irradiation module according to FIG. 2 or 3;

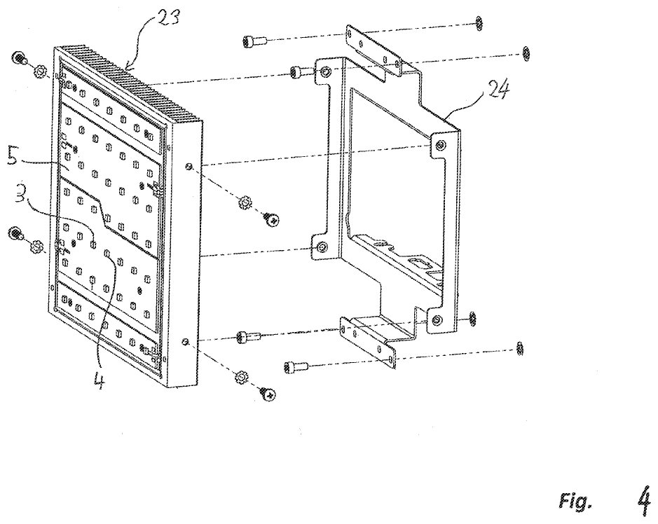

FIG. 5 a cross-sectional view through part of an irradiation module according to FIG. 2, 3 or 4; and

FIG. 6 a cross-sectional view of a further embodiment of an irradiation module.

FIG. 1 shows a body irradiation device 1. It comprises an exposure tunnel 16 in which a user can lie in order to be irradiated with actinic radiation.

Preferably, the exposure tunnel 16 is closed, substantially by an upper part 26 of the body irradiation device 1 being pivoted toward a lower part 27 of the body irradiation device 1, after the user has entered the exposure tunnel 16.

The lower part 27 of the body irradiation device 1 comprises an at least substantially transparent surface 17, under which irradiation modules 2 with LED radiation sources 3, 4 are arranged. The surface 17 thereby separates the exposure tunnel 16 into an inner space 19 and an outer space 20 in which the irradiation modules 2 are arranged. The outer space 20 can in particular be cooled by an airflow.

FIG. 2 shows a top plan view of an irradiation module 2 of a body irradiation device 1.

The irradiation module 2 comprises a plate 6 on which planoconvex lenses 7, 8 are arranged. These lenses 7, 8 are preferably integrally bonded to the plate 6. Further preferably, they are bonded to the opposite side 9 of the plate 6 from a carrier 5 (not shown) and in particular arranged on the surface of this side 9. The irradiation module 2 further comprises spacers 21a, 21b, 21c, 21d, the upper ends or fixing means of which are visible in FIG. 2.

Furthermore, part of a mounting frame 24 of the irradiation module 2 is visible in FIG. 2, wherein screws (no reference numeral) are inserted into holes of this mounting frame in order to fix the mounting frame 24 to the body irradiation device 1.

FIG. 3 shows an exploded view of the irradiation module 2 according to FIG. 2.

The plate 6 shown in FIG. 2 is shown in the exploded view. The plate 6 is in each case preferably a glass plate, to the surface 9 of which the lenses 7, 8 are integrally bonded, in particular glued by means of an adhesive.

Preferably, the surface 9 of the plate 6 is satinized. Through the use of the adhesive, said satinizing is preferably eliminated in the area of the lenses 7, 8 so that the interfaces between the flat surfaces of the lenses 7, 8 and the plate 6 become transparent. A plastic plate 11 is preferably arranged on the side of the plate 6 facing a printed circuit board 5, which preferably forms the carrier 5. It preferably has recess 12, 13 which are cut from those areas of the plastic plate 11 corresponding to the arrangement of lenses 7, 8 on the plate 6.

Alternatively, the surface 10 of the side of the plate 6 facing the carrier 5 can also be satinized.

The plastic plate 11 preferably consists of or comprises a fluorescent material which can be activated to glow by the radiation from LED radiation sources 3, 4 on the circuit board 5.

The plastic plate 11 is fixed and aligned or respectively positioned on the plate 6 preferably by a fixing plate 22 which surrounds the plastic plate 11 at its end faces.

Arranged on the right side of the exploded view according to FIG. 3 is a printed circuit board on which the LED radiation sources 3, 4 are arranged, affixed and electrically connected.

A cooling device 23, cooling fins 23 in the present case, is arranged on the opposite side of the printed circuit board 5 from the plate 6. A thermal paste is preferably introduced between the cooling device 23 and the circuit board 5.

The irradiation module 2 can preferably be fixed by the support frame 24. In the present embodiment, the support frame 24 bears the cooling means 23 and the printed circuit board 5 affixed to the cooling means 23. The plate 6, the fixing plate 22 and thus also the plastic plate 11 are affixed to the cooling device 22 by means of screws (no reference numeral). Further preferably, however, they can also be fixed directly to the support frame 24. Spacers 21a, 21b, 21c, 21d, which set a defined distance, are provided between the plate 6 and/or the plastic plate 11 on the one side and the printed circuit board 5. They can either be arranged between the fixing plate 22 and the plate 6 and/or between the fixing plate 22 and the circuit board 5 or cooling device 23 or support frame 24, depending on how the securing of the screws is designed.

FIG. 4 shows a further exploded view of the aggregate of the printed circuit board 5 and the support frame 24 shown in FIG. 3.

As can be seen from this figure, the support frame 24 can be fixed to the cooling device 23 in this embodiment by means of screws.

FIG. 5 shows a cross-sectional view of an irradiation module 2 according to FIGS. 2 to 4. FIG. 5 is schematic such that it differs slightly from the actual embodiment of FIGS. 2 to 4. Nor is the cooling device 23 shown in FIG. 5, although it can be arranged on the underside of the carrier 5.

From top to bottom, the individual elements of the irradiation module 2 in FIG. 5 are arranged as follows: The planoconvex lenses 7, 8 are integrally bonded to the glass plate 6 by their flat side. The opposite side 9 of the glass plate from the carrier 5 is thereby preferably satinized. The plastic plate 11, which exhibits recesses 12, 13 in the region of the lenses 7, 8 is arranged below the glass plate 6. The side 10 of the glass plate 6 facing the carrier 5 is preferably mirrored. Alternatively, the glass plate 6 can also not exhibit any sanitizing and/or any mirroring. Further alternatively or additionally, the respective satinizing and/or mirroring can be arranged on the respective other side of the glass plate 6.

The plastic plate 11 preferably comprises fluorescent material able to be activated by the LED radiation sources 3, 4. The radiation sources 3, 4 are each arranged in the region of an optical axis of the lenses 7, 8 so that a large portion of the radiation emitted by the LED radiation sources 3, 4 enters the lenses 7, 8 through the recess 12, 13 and can be at least substantially collimated there.

With respect to the carrier 5 with the LED radiation sources 3, 4 there are substantially two different embodiments, both of which are depicted in FIG. 5.

Normally, however, only one of the two embodiments will be implemented in a single irradiation module 2. The representation of both embodiments as depicted here serves in particular for illustrative purposes.

The carrier 5 is held at a defined distance from the plastic plate 11 and/or the glass plate 6 here by the spacers 21a, 21b, 21c.

The varying embodiments differ by whether the carrier 5 is formed by a printed circuit board. In this case, as shown on the left side, a substrate 18 is arranged on the circuit board 5, onto which an LED chip 14 configured to emit the radiation can ultimately be arranged.

Alternatively, the carrier 5 itself can also be designed as a substrate. In this case, the LED chip 14 or, as shown on the right in FIG. 5, LED chips 14, 15 if the LED radiation source has two LED chips, is/are arranged directly on the carrier 5. The LED radiation sources 3, 4 and the printed circuit board 5 are thereby designed in a manner known from the prior art.

FIG. 6 shows a cross section of a further embodiment of an irradiation module 2.

The embodiment shown in FIG. 6 substantially differs from the embodiment shown in FIG. 5 by there only being one plate 6 in the upper region of the irradiation module 2. This plate 6 is preferably designed as a plastic plate.

The recesses 12, 13 exhibit two bore diameters in this embodiment. The lower bore diameter is thereby reduced compared to the upper bore diameter such that noses, protrusions or shoulders are formed which in each case form a seat 25, 26 for the lenses 7, 8. Like the lenses 7, 8 this seat is preferably rounded.

The lenses 7, 8 are preferably integrally bonded to the seats 25, 26 particularly by means of an adhesive. The lenses 7, 8 are thereby also aligned or respectively centered via the seats 25, 26 in such a way that the flat surface of the lenses 7, 8 preferably runs at least substantially parallel to the carrier 5. Preferably, the side 10 of the plastic plate 6 facing the carrier 5 is mirrored in this embodiment and the plastic plate 6 also further preferably comprises fluorescent material.

Preferably, different areas of the plastic plate 6, 11 of FIGS. 5 and 6 can be provided with fluorescent material which fluoresces in different colors. Preferably, it can be provided for the fluorescent color to be adapted to the emission spectrum of the respective LED chip of the LED radiation source 3, 4.

With respect to the LED radiation sources 3, 4 and the carrier 5, that which has already been stated with regard to FIG. 5 applies. Two differing embodiments of the LED radiation sources 3, 4 are again depicted in FIG. 6, these would also be suitable with respect to the embodiment according to FIG. 5 (and vice versa). Here, one LED chip 14 is arranged directly on a substrate 18 on the left side; on the right side, the carrier 5 would be the printed circuit board. There is an additional substrate 18 on which two LED chips, preferably having a differing radiation spectrum, are arranged.

It should be noted that the embodiments are only examples which are in no way intended to limit the scope of protection, application and configuration. Rather, the foregoing description is to provide the person skilled in the art with a guideline for implementing at least one embodiment, whereby various modifications can be made, particularly as regards the function and arrangement of the described components, without departing from the scope of protection as results from the claims and combinations of features equivalent to same.

### REFERENCE NUMERALS

1 body irradiation device
2 irradiation module
3, 4 LED radiation source
5 carrier
6 plate
7, 8 lens
9 plate side opposite from the carrier
10 plate side facing the carrier
11 plastic plate
12, 13 recess
14 first LED chip
15 second LED chip
16 exposure tunnel
17 first surface
18 substrate
19 inner space
20 outer space
21a, 21b, 21c, 21d spacer
22 fixing plate
23 cooling device
24 support frame
25, 26 seat
27 lower part
28 upper part

1. A body irradiation device (1) for use of directional actinic radiation on a living organism, in particular a human being, which comprises at least one irradiation module (2), and wherein the at least one irradiation module (2) comprises:
at least two LED radiation sources (3, 4) which generate the actinic radiation and are arranged on a common carrier (5);
a plate (6) which spans the at least two LED radiation sources (3, 4), wherein the plate (6) is spaced apart from the carrier (5);
at least two planoconvex optical lenses (7, 8) which are integrally bonded to the plate (6) such that the flat surfaces of the lenses (7, 8) face the carrier (6), and wherein each lens (7, 8) is configured and arranged in such a way as to at least substantially collimate or direct radiation emitted by one of the LED radiation sources (3, 4).

2. The body irradiation device (1) according to claim 1, wherein at least one plate side (9), in particular the opposite side (9) of the plate (6) from the carrier (5), is satinized.

3. The body irradiation device (1) according to claim 1 or 2, wherein one side of the plate (6), in particular the side (10) of the plate (6) facing the carrier (5), is coated, particularly with a mirroring for, in particular exclusively, radiation in the visible spectrum.

4. The body irradiation device (1) according to one of the preceding claims, wherein the at least two planoconvex lenses (7, 8) are glass-molded lenses.

5. The body irradiation device (1) according to one of the preceding claims, wherein the plate (6) is a glass plate.

6. The body irradiation device (1) according to one of the preceding claims, wherein the at least one irradiation module (2) further comprises:
an at least partially transparent plastic plate (11) which covers the plate (6), in particular on the side (10) facing the carrier (5), and has recesses (12, 13) in the region of the at least two lenses (7, 8).

7. The body irradiation device (1) according to one of claims 1 to 4, wherein the plate (6) is a partially transparent plastic plate.

8. The body irradiation device (1) according to claim 7, wherein the plastic plate (6) comprises, in particular round, recesses (12, 13) in the region of the at least two lenses (7, 8) which are designed such that the at least two lenses (7, 8) are able to be integrally bonded to the plastic plate (6).

9. The body irradiation device (1) according to claim 8, wherein the recesses (12, 13) each form a seat (25, 26) for the flat side of the at least two lenses (7, 8).

10. The body irradiation device (1) according to one of the preceding claims, wherein the plate (6) and/or the plastic plate (11) comprises a fluorescent material, in particular is coated with the fluorescent material.

11. The body irradiation device (1) according to claim 10, wherein a first region of the plate (6) and/or the plastic plate (11) contains violet fluorescent material opposite from a UV-A LED radiation source, wherein a second region of the plate and/or the plastic plate (11) contains red fluorescent material opposite from a red LED radiation source, and/or wherein a third region of the plate and/or the plastic plate (11) contains yellow fluorescent material opposite from a UV-B LED radiation source.

12. The body irradiation device (1) according to one of the preceding claims, wherein the at least two planoconvex lenses (7, 8) are glass lenses.

13. The body irradiation device (1) according to one of the preceding claims, wherein at least one of the LED radiation sources (3; 4) emits UV-A radiation and the lens (7; 8) associated with this LED radiation source for collimating the radiation consists of borosilicate glass.

14. The body irradiation device (1) according to one of the preceding claims, wherein at least one of the LED radiation sources (3; 4) emits UV-B radiation and the lens (7; 8) associated with this LED radiation source for collimating the radiation consists of quartz glass.

15. The body irradiation device (1) according to one of the preceding claims, wherein the integral bond is a UV-curing adhesive.

16. The body irradiation device (1) according to one of the preceding claims, wherein at least one of the LED radiation sources (3) comprises a first LED chip (14) having a first radiation spectrum and a second LED chip (15) having a second radiation spectrum differing from the first radiation spectrum.

17. The body irradiation device (1) according to one of the preceding claims, wherein at least one first LED radiation source (3) exhibits a first radiation spectrum and wherein at least one second LED radiation source (4) exhibits a second radiation spectrum.

18. The body irradiation device (1) according to claim 11, particularly in conjunction with one of claims 12 to 17, wherein the first region of the plate (6) is arranged around a lens (7) which collimates light of the first LED radiation source (3) or the first LED chip (14), and wherein the second region of the plate (6) is arranged around a lens (8) which collimates light of the second LED radiation source or the second LED chip.

19. The body irradiation device (1) according to claim 17 or 18, wherein the first LED chip (14) and/or the first LED radiation source (3) emits a UV-A or UV-B spectrum and the second LED chip (15) and/or the second LED radiation source (4) emits in the red spectrum.

20. The body irradiation device (1) according to one of the preceding claims, wherein the irradiation module (2) further comprises a cooling device (23) configured to cool the carrier (5) at least on the opposite side from the at least two LED radiation sources (3, 4).

21. The body irradiation device (1) according to one of the preceding claims further comprising:
an exposure tunnel (16) capable of surrounding the living organism, wherein the exposure tunnel (16) is formed from at least one lower part (27) of the body irradiation device (1) having a surface (17) made from a material which is substantially transparent to the actinic radiation and at least one upper part (26) of the body irradiation device (1), wherein the surface (17) separates an inner space (19), in which the living organism can be exposed to the actinic radiation, from an outer space (20) in which at least one LED irradiation module (2) is mounted in such a way as to emit actinic radiation through the surface (17), and wherein the upper part (26) also has at least one LED irradiation module (2) mounted in such a way as to emit actinic radiation into the inner space (19).

22. The body irradiation device (1) according to one of the preceding claims, wherein the at least one irradiation module (2) further comprises: spacers (21a, 21b, 21c, 21d) between the plate (6) and the carrier (5) which keep the plate (6) and the carrier (5) at a defined distance.
